(19) United States
(12) Patent Application Publication
(10) Pub. No.: US 2025/0382563 A1
(43) Pub. Date: Dec. 18, 2025

(54) BIOPROCESSING SYSTEM AND METHODS

(71) Applicant: Donaldson Company, Inc., Bloomington, MN (US)

(72) Inventors: Vincent J. Fischer, Plougourvest (FR); Daria V. Nikolaeva, Oud-Heverlee (BE)

(21) Appl. No.: 19/237,806

(22) Filed: Jun. 13, 2025

Related U.S. Application Data
(60) Provisional application No. 63/660,266, filed on Jun. 14, 2024.

(51) Int. Cl. C12M 1/34; C12M 1/00; C12M 1/26

(52) U.S. Cl. CPC C12M 41/36; C12M 33/14; C12M 39/00

(57) ABSTRACT

A bioprocessing system including a contained bioprocessing environment, a separator assembly, a first retentate flow line, a second retentate flow line, and a retentate system outlet flow line. The separator assembly includes a separator inlet, a retentate outlet, and a permeate outlet. The separator assembly is configured to be fluidly coupled to the contained bioprocessing environment. The first retentate flow line is configured to extend from the retentate outlet to the separator inlet. The second retentate flow line is configured to extend from the retentate outlet to the contained bioprocessing environment. The retentate system outlet flow line is configured to extend from the retentate outlet to a system outlet.

### RELATED APPLICATIONS

This application claims the benefit of U.S. Provisional Patent Application No. 63/660,266 filed on Jun. 14, 2024 titled BIOPROCESSING SYSTEM AND METHODS, which is hereby incorporated by reference in its entirety.

### TECHNOLOGICAL FIELD

The present disclosure is generally related to bioprocessing systems and methods.

### SUMMARY

The present disclosure relates to a system for processing biomaterial. The present disclosure also relates to a method for processing biomaterial. The present disclosure further relates to systems and method for processing biomaterial from cultured cell suspension, from microorganism suspension, and from any combination thereof.

Bioprocessing systems are widely applicable in various industries, including food, beverage, pharmaceutical, cosmetic, artificial food industry, cellular agriculture, environmental, and other industries. Contained bioprocessing environments such as bioreactors and fermenters are vessels that are used for growing and expanding microorganisms, maintaining cell activity, and supporting cell cultures under controlled conditions to carry out biochemical processes. In some cases, inactivation of cells or sterilization is carried out in the contained bioprocessing environment such as in water treatment. Further, in general, contained bioprocessing environment can hold suspension cultures or adhesion cultures. A suspension culture is a suspension of cells in a culture media, where a “culture media” is a media within which cells are suspended or to which cells are adhered to (e.g., water, sugars, proteins, cell byproducts, etc.). A cell byproduct is a substance generated during cellular metabolism that is produced or released by the cell during its growth, metabolism, or after cell lysis. An adhesion culture is where adherence of cells to a surface area supports cell proliferation.

A contained bioprocessing environment such as a bioreactor, reactor, or fermenter is the site of cell proliferation, biomolecule production or transformation, and cell death. Relevant considerations for such systems include, as examples, maximizing cell proliferation, removal of dead cells and other pollutants, addition of fresh media containing nutrients and/or cell proliferation/growth factors, and extraction of cell byproducts. Renewal of culture media from the bioreactor (i.e., removing some culture media and replacing it with fresh culture media) may upset the rate of cell division and cell proliferation, because there are then less cells proliferating within the contained bioprocessing environment. Thus, removal and/or renewal of culture media is generally performed during maximum cell proliferation in the contained bioprocessing environment, to avoid upsetting the culture within the environment. When the culture within the contained bioprocessing environment is mostly made up of lower-proliferating cells (e.g., cells in the beginning or end of their individual life cycle), the cell proliferation within the contained bioprocessing environment may be lower than when the contained bioprocessing environment is mostly made up of healthy proliferating cells. Thus, removal of culture media from the contained bioprocessing environment for processing can more heavily affect the concentration of the cells within the environment when the culture within the environment is relatively slower-proliferating. That is, if the culture is relatively slower-proliferating, removal of culture media can affect the overall cell concentration more heavily than when the culture is made up of relatively higher-proliferating cells, at least because relatively lower-proliferating cells do not multiply as quickly and cannot replenish the overall cell concentration as quickly. When the culture is made up of relatively higher-proliferating cells, the cells in the culture can multiply relatively quickly, and thus, removal of some of the culture media does not affect the overall cell concentration as heavily since the culture can replenish faster.

In many research and development applications and industrial applications, contained bioprocessing environments are used in conjunction with filters to create a bioprocessing system. In some implementations one or more filters can be used to filter the culture media removed from the contained bioprocessing environment. In some implementations one or more filters can be used to remove undesirable components, and/or retain, accumulate, or concentrate target components. In some implementations, one or more filters can be used to separate target components from other components. Desirable/target and undesirable components may be defined by the specific application of the bioprocessing system. For example, the target components may be cells, particles produced by cells, or other components within the fluid.

A current limitation to success and competitiveness in the application of culture medias and bioprocessing systems to create products is the difficulty in maintaining the desired concentration of cells in the contained bioprocessing environment, and the difficulty in maintaining the overall environment of the contained bioprocessing environment during culture media removal from the environment and during material addition to the environment. These difficulties further contribute to the lack of continuous bioprocessing. Instead, most applications use batch bioprocessing that targets maximum cell proliferation for cell harvesting, and culture media removal is not refreshed when the cells are harvested, followed by ending the batch application and re-starting a new batch with fresh ingredients and a clean, or new, bioprocessing system. Thus, batch bioprocessing is generally time-intensive and expensive, because batch bioprocessing requires waiting for an ideal culture media and cell concentration, harvesting a relatively small batch, and stopping the application. Each batch may take days, weeks, or months (e.g., 3 to 4 weeks, 3 to 4 months, etc.) to complete.

Improvements to these systems and methods regarding the ideal cell proliferation rate and cell concentration within the contained bioprocessing environment are desired. It may be desirable to continuously or semi-continuously remove cell product, without substantially affecting the volumetric production rate within the contained bioprocessing environment. “Volumetric production rate” is the production rate of a cell product by unit volume. “Cell product” is defined as the target/desirable components from the bioprocessing environment, which can include multiple components. Example cell products can include the cells themselves (which may include one or more types of cells) and/or one or more cell byproducts. It may further be desirable to add media containing nutrients and/or growth factors without substantially affecting the culture within the contained bioprocessing environment. It may further be desirable to enable the re-use of spent media such as by removing cell byproducts instead of disposing of spent media after a single use, which can be costly.

In one aspect, the present disclosure describes continuous bioprocessing. It is noted that “continuous” is not used herein to necessarily mean “constantly” Rather, “continuous” is generally used herein to refer to a relatively prolonged period of time. “Continuous bioprocessing” is used herein to distinguish from “batch processing” that is described above. Continuous bioprocessing refers to processes where the duration of productivity is expanded compared to batch processing by modifying the suspension media during bioprocessing to maintain and extend the productivity of the suspension media. For example, components of the suspension media that interfere with productivity of the suspension media can be constantly or incrementally removed from the suspension media. As another example, components of the suspension media that contribute to the productivity of the suspension media can be incrementally or constantly added to the suspension media when they are depleted. Continuous bioprocessing allows cell proliferation to be maintained at a desirable proliferation rate for longer periods of time than batch bioprocessing. Further, the present disclosure describes removal of cell products from the bioprocessing system without substantially affecting the culture media or the cell concentration within the contained bioprocessing environment, which allows the cell proliferation to be maintained at a desirable proliferation rate for relatively longer periods of time. Some embodiments relate to a separator system that is configured for continuous collection of cell product(s), which means that cell products can be collected constantly or incrementally over a relatively longer period of time compared to batch processing.

Some embodiments of the present technology relate to a bioprocessing system. The bioprocessing system has a separator assembly having a separator inlet, a retentate outlet, and a permeate outlet. The separator assembly is configured to be fluidly coupled to a contained bioprocessing environment via a bioprocessing environment flow line. A first retentate flow line is configured to be fluidly coupled to the retentate outlet and the separator inlet. A second retentate flow line is configured to be fluidly coupled to the retentate outlet and the contained bioprocessing environment. A retentate system outlet flow line is configured to be fluidly coupled to the retentate outlet and a system outlet.

In some such embodiments, a retentate valve is operatively coupled to the second retentate flow line. Additionally or alternatively, the separator assembly has a diafiltration system. Additionally or alternatively, a washing line is fluidly coupled to the separator inlet. Additionally or alternatively, the bioprocessing system is configured to be cleaned-in-place. Additionally or alternatively, the separator assembly has components that are reusable and washable. Additionally or alternatively, the separator assembly excludes single use components. Additionally or alternatively, a cleaning solution tank is in selective communication with the separation assembly.

Additionally or alternatively, a steam generator is configured for selective fluid communication with the cleaning solution tank. Additionally or alternatively, the separator assembly includes a tangential flow filter. Additionally or alternatively, the separator assembly includes a hydrodynamic separator. Additionally or alternatively, the separator assembly includes a membrane. Additionally or alternatively, a controller is operatively coupled to the first retentate flow line, the second retentate flow line, and the retentate system outlet flow line, and the controller is configured to control a flow through each flow line. Additionally or alternatively, the controller is configured to control the flow through each line to maintain a cell concentration of the contained bioprocessing environment.

Additionally or alternatively, the cell concentration of the contained bioprocessing environment remains substantially constant when a cell product is removed from the bioprocessing system by way of the retentate system outlet flow line. Additionally or alternatively, when the controller allows flow through the retentate system outlet flow line, the controller does not allow flow through the second retentate flow line. Additionally or alternatively, a connecting flow line is fluidly coupled to the first retentate flow line and the second retentate flow line. The connecting flow line is fluidly coupled to the first retentate flow line and the retentate system outlet flow line.

Additionally or alternatively, a connecting line valve is operably coupled to the connecting flow line and controllable to open or close the connecting flow line. Additionally or alternatively, the first retentate flow line, the second retentate flow line, and the retentate system outlet flow line are each fluidly coupled to a four-way connector, and wherein the four-way connector is fluidly coupled to the retentate outlet. Additionally or alternatively, the system includes the contained bioprocessing environment. Additionally or alternatively, the contained bioprocessing environment is a bioreactor. Additionally or alternatively, the system is configured to continuous bioprocessing. Additionally or alternatively, the system is configured for continuous collection of cell product.

Some embodiments of the present technology relate to a method for operating a bioprocessing system. Fluid flow is directed from a contained bioprocessing environment to a separator inlet of a separator assembly. The separator assembly separates the fluid into a permeate and a retentate. Retentate flow is selectively directed from a retentate outlet of the separator assembly to two or more in the group consisting of: a separator inlet of the separator assembly, the contained bioprocessing environment, and a first system outlet.

In some such embodiments, directing flow of the retentate from the retentate outlet to the separator inlet includes directing flow through a first retentate flow line. Additionally or alternatively, directing flow of the retentate from the retentate outlet to the contained bioprocessing environment includes directing flow through a second retentate flow line. Additionally or alternatively, a retentate valve operatively coupled to the second retentate flow line is controlled using a controller operatively coupled to the retentate valve. Additionally or alternatively, directing flow of the retentate from the retentate outlet to a first system outlet includes directing flow through a retentate system outlet flow line.

Additionally or alternatively, the method includes directing flow from the contained bioprocessing environment to the separator inlet. Additionally or alternatively, flow is directed from the contained bioprocessing environment through the second retentate flow line and bypasses the separator assembly. Additionally or alternatively, the fluid is diafiltrated using the separator assembly. Additionally or alternatively, a washing fluid flow is directed from a washing line to the separator inlet. Additionally or alternatively, flow of the permeate is directed from a permeate outlet of the separator assembly to the washing line. Additionally or alternatively, a cell concentration of the contained bioprocessing environment is maintained to remain substantially constant when a cell product is removed from the first system outlet.

Additionally or alternatively, when a cell product is removed from the bioprocessing system, the volumetric production rate is not affected by the removal of the cell product. Additionally or alternatively, a clean-in-place system cleaning procedure is initiated. Additionally or alternatively, a sterilization system cleaning procedure is initiated. Additionally or alternatively, the separator assembly includes a tangential flow filter. Additionally or alternatively, the separator assembly includes a hydrodynamic separator. Additionally or alternatively, the separator assembly includes a membrane separator. Additionally or alternatively, the method includes directing flow from the contained bioprocessing environment through a portion of the first retentate flow line towards the second retentate flow line. Additionally or alternatively, the method is a substantially continuous bioprocessing method. Additionally or alternatively, the method includes substantially continuous collection of cell product.

Some embodiments of the present technology relate to a bioprocessing system having a contained bioprocessing environment, a separator assembly, a first retentate flow line, a second retentate flow line, a retentate system outlet flow line, a connecting flow line, a loop breaker valve, a first retentate valve, and a second retentate valve. The separator assembly includes a separator inlet, a retentate outlet, and a permeate outlet. The separator inlet is configured for fluid communication with the contained bioprocessing environment. The first retentate flow line is configured to be fluidly coupled to the retentate outlet and the separator inlet. The second retentate flow line configured to be fluidly coupled to the first retentate flow line and the contained bioprocessing environment. The retentate system outlet flow line configured to be fluidly coupled to the first retentate flow line and a system outlet. The connecting flow line is configured to be fluidly coupled to the first retentate flow line, the second retentate flow line, and the retentate system outlet flow line. The loop breaker valve is operatively coupled to the first retentate flow line. The first retentate valve is operatively coupled to the second retentate flow line. The second retentate valve is operatively coupled to the retentate system outlet flow line, whereby bypassing the separator assembly is enabled.

In some such embodiments, the loop breaker valve is positioned downstream of the connection flow line. Additionally or alternatively, each of the loop breaker valve, first retentate valve, and the second retentate valve have a fully open position, a fully closed position, and an intermediate position between the fully open and fully closed position. Additionally or alternatively, the system is configured for continuous bioprocessing. Additionally or alternatively, the system is configured for continuous collection of cell product.

The above summary is not intended to describe each embodiment or every implementation. Rather, a more complete understanding of illustrative embodiments will become apparent and appreciated by reference to the following Detailed Description and claims in view of the accompanying figures of the drawing.

### BRIEF DESCRIPTION OF DRAWINGS

FIG. 1 is a schematic representation of a bioprocessing system according to one embodiment.

FIG. 2 is a diagram of a bioprocessing system in accordance with the embodiment of FIG. 1.

FIG. 3 is an exemplary method of using a bioprocessing systems as described herein.

The present technology may be more completely understood and appreciated in consideration of the following detailed description of various embodiments in connection with the accompanying drawings.

The figures are rendered primarily for clarity and, as a result, are not necessarily drawn to scale. Moreover, various structure/components, including but not limited to fasteners, electrical components (wiring, cables, etc.), and the like, may be shown diagrammatically or removed from some or all of the views to better illustrate aspects of the depicted embodiments, or where inclusion of such structure/components is not necessary to an understanding of the various exemplary embodiments described herein. The lack of illustration/description of such structure/components in a particular figure is, however, not to be interpreted as limiting the scope of the various embodiments in any way.

### DETAILED DESCRIPTION

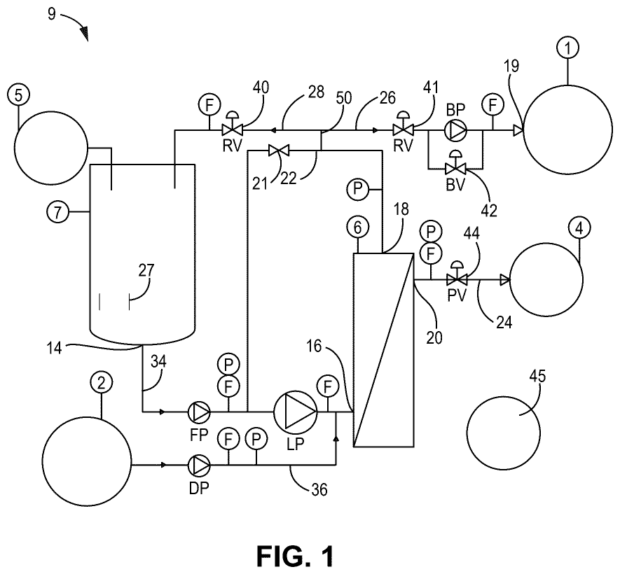

In one or more embodiments, and as illustrated in FIG. 1, a bioprocessing system 9 may include a contained bioprocessing environment 7 and a separator assembly 6. The contained bioprocessing environment 7 may be any appropriate and known bioreactor, fermenter, reactor, and/or cell cultivation apparatus. The separator assembly 6 is defined as any assembly that is configured to separate whole cells from surrounding culture media or separate components within the culture media. The components of the cell and media solution can include water, sugars, biomolecules, proteins, amino acids, vitamins, growth factors, cell byproducts (such as ammonia), dead cells, live cells, and the like, etc.

The separator assembly 6 may include separator components including but not limited to a tangential flow filter, a separation membrane, a hydrodynamic separator, an ion exchange separator, a hollow filter, a ceramic filter, membrane chromatography system, continuous centrifuge, and the like, or any combination thereof. In some embodiments, the assembly 6 is reusable. In some embodiments one or more separator components are reusable. In some embodiments, one or more separator components are single use. In some embodiments, the one or more separator components are exchangeable. The separator assembly 6 may include multiple separator components arranged in parallel and/or series to achieve the desired separation of the components in the cell and media solution. A separator assembly 6 including separation components that are arranged in series may advantageously allow for refinement of the separation of the various components. A separator assembly 6 including separation components that are arranged in parallel may advantageously increase the separation capacity of the system.

In some embodiments the separator assembly 6 can include a tangential flow filter. In some embodiments the separator assembly 6 can include a separator membrane such as, for example, chromatography membranes such as Purexa™ by Donaldson, Inc. in Bloomington, Minnesota, USA. In some embodiments the separator assembly 6 can include hollow fiber filter. Some example hollow fiber filters can have an inner diameter of 1 mm to 5 mm. In some embodiments the separator assembly 6 can include a ceramic filter. In some embodiments the separator assembly 6 can include a plate and frame membrane system incorporating a polymeric membrane. In some embodiments the separator assembly 6 can include a hydrodynamic separator system. In various embodiments, the separation performance can range from 1 to 200 LMH (liters per square meter per hour).

It is noted that the specific performance characteristics and materials of the separator components within the separator assembly 6 employed will generally be dependent on the particular implementation for which the bioprocessing environment is used. Membrane materials such as PES, PDV, cellulose acetate can be used depending on the sensitivity of the cell product and the demands of the process. For example, hollow fiber membranes made of polyethersulfone (PES) or polyvinylidene fluoride (PVDF) with 0.2 μm pores may be used to obstruct passage of cells like CHO or HEK293 while letting some proteins they produce flow through. Membranes with 0.45 μm pores may be used to let viral vectors like lentivirus or AAV pass through while obstructing passage of cells and debris. In some examples, depth filters with pore sizes between 0.1 and 1.0 μm may be employed to help purify the culture media by removing debris and aggregates. In ultrafiltration applications, membranes having molecular weight cutoffs (MWCOs) ranging from 10 kDa (kilodaltons) to 100 kDa may be employed. Such membranes may be applicable to concentrating therapeutic proteins like monoclonal antibodies while removing small impurities and salts. In some implementations separation materials having pore sizes of about 20 nanometers (roughly 0.02 μm) may be used. Such separation materials may be applicable to, for example, trapping potential viral contaminants without obstructing passage of a protein product. In some implementations membranes having pore sizes from 0.2-0.5 μm may be used. Such membrane may have applicability in, for example, cell and gene therapy manufacturing, where cells themselves are the cell product. Such membranes may be used in relatively gentle filtration methods employing low shear pumps in the systems to keep cells alive and in the culture media while refreshing the culture media. In some implementations, microfiltration membranes having a pore size of 0.1-0.3 μm may be used. Such membranes may be applicable to media or buffer sterilization. Sterilizing grade filters may be used to remove bacteria and particulates. For example, such filters may be used to purify injectable biologics.

The contained bioprocessing environment may include a bioprocessing outlet 14. The separator assembly 6 may include a separator inlet 16, a retentate outlet 18, and a permeate outlet 20. In some embodiments the retentate may include one or more desirable components in a product of a bioprocessing system. In some embodiments, permeate may include one or more undesirable components in a product of a bioprocessing system. In some embodiments, permeate may also include one or more desirable components in a product of a bioprocessing system. The retentate outlet 18 may allow flow of retentate therethrough, and the permeate outlet 20 may allow flow of permeate therethrough. The construction of the separator assembly may be such that permeate does not exit the retentate outlet 18, and retentate does not exit the permeate outlet 20. The separator assembly 6 may be configured to be fluidly coupled to the contained bioprocessing environment 7. Fluid couplings may include one or more flow line(s) such as flexible or rigid conduits that allow flow of a fluid therethrough. Fluid couplings may include one or more valves. Valves may be one-, two-, three- or four-way valves. Valves may be controlled by a controller 45 and/or be manually controlled. Fluid couplings may be direct (e.g., point A to point B) or may be indirect (e.g., point A to point C, D, etc., to point B).

The separator assembly 6 may be configured to be aseptically coupled to the contained bioprocessing environment 7. Alternatively, the separator assembly 6 may be configured to be sterilely coupled to the contained bioprocessing environment 7. Such aseptic or sterile couplings avoid contaminating the bioprocessing system, which may adversely affect the system and the final product of the application. In many cases, such as in batch bioprocessing systems, a new separator assembly may be coupled to a contained bioprocessing environment after each batch application is complete, requiring many separator assemblies to complete many batches. There are also more chances of contamination in such batch bioprocessing systems. In one or more embodiments herein, and as illustrated in FIG. 1, the separator assembly 6 may be coupled to the contained bioprocessing environment 7 and then used continuously, avoiding contamination and becoming more cost-efficient. In one or more embodiments, the bioprocessing system may includes single use components that are sterile or sterilizable prior to use. In one or more embodiments, the bioprocessing system may include reusable components that are sterilizable prior to reuse.

The bioprocessing system 9 may further include a first retentate flow line 22. The first retentate flow line 22 may be configured to fluidly couple the retentate outlet 18 and the separator inlet 16. The first retentate flow line 22 may be configured to allow flow of a retentate therethrough. A loop pump LP is configured to be in fluid communication with the first retentate flow line 22 to drive fluid flow through the first retentate flow line 22. In various embodiments a controller 45 is in communication with the loop pump LP to control fluid flow through the flow line. The bioprocessing system 9 may further include a second retentate flow line 28. The second retentate flow line 28 may be fluidly coupled to the retentate outlet 18 and the contained bioprocessing environment 7. The second retentate flow line 28 may be configured to allow flow of a retentate therethrough. The bioprocessing system 9 may further include a retentate system outlet flow line 26. The retentate system outlet flow line 26 may be configured to fluidly couple the retentate outlet 18 and a system outlet 19. Such fluid coupling may allow continuous or discrete flow of the retentate. The retentate system outlet flow line 26 may be configured to allow flow of a retentate therethrough. In various embodiments, a bleed pump BP is configured to be in fluid communication with the retentate system outlet flow line 26 to drive fluid flow through that flow line. In various embodiments a controller 45 is in communication with the bleed pump BP to control fluid flow through the retentate system outlet flow line 26.

As illustrated in FIG. 1, each of flow lines 22, 28, and 26 may be fluidly coupled to the retentate outlet 18 and allow flow of the retentate therethrough. Each of flow lines 22, 26, and 28 may be completely separate from one another (e.g., as illustrated in FIG. 1, flow line 22 ends before flow lines 26, 28 begin). In alternative embodiments, each of flow lines 22, 26, and 28 may share a common section of flow line prior to separating (e.g., not shown, a four-way flow line connector between a common flow line extending from the retentate outlet 18, and each of flow lines 22, 26, and 28).

As illustrated in FIG. 1, the bioprocessing system allows for retentate to flow from the retentate outlet 18 to one or more of the separator inlet 16, the contained bioprocessing environment 7, and the system outlet 19. In one or more embodiments, the retentate is configured to flow to one of the three locations. In one or more embodiments, the retentate is configured to flow to two of the three locations simultaneously. In one or more embodiments, the retentate is configured to flow to all three locations simultaneously.

The bioprocessing system may further include a first retentate valve 40 operatively coupled to the second retentate flow line 28. The first retentate valve 40 may be controlled to be fully open, fully closed, or in an intermediate configuration between fully opened and fully closed to define a particular fluid flow rate. Thus, retentate flow to the contained bioprocessing environment 7 may be controlled. In some implementations, the user or a controller 45 can selectively engage the first retentate valve 40 to direct retentate into the contained bioprocessing environment 7. Such routing of the retentate flow may be desirable where data analysis identifies a relatively high concentration of relatively low-proliferation cells (or a relatively low concentration of relatively high-proliferation cells), and further cell proliferation is desirable. In some implementations, the user or a controller 45 can selectively engage the first retentate valve 40 to obstruct retentate from flowing into the contained bioprocessing environment 7. Such routing of the retentate flow may be desirable where data analysis identifies that the cells are a relatively high concentration of relatively high-proliferation cells that are ready for extraction from the system. Sensors can be configured to take inline measurements of cell count and/or other parameters, which can be processed by the controller 45 to assess cell viability. Such automation may advantageously streamline system processes. In such an example, the retentate flow may be routed through the retentate system outlet 26 flow line. In some embodiments, the retentate flow may be routed to a collection vessel, container, bag, etc. In some embodiments, the retentate flow may be routed to a further processing step within, or outside of, the bioprocessing system 9.

The bioprocessing system may further include a second retentate valve 41 operatively coupled to the outlet flow line 26. The second retentate valve 41 may be controlled to be fully open, fully closed, or in an intermediate configuration between a fully opened and fully closed position to define a particular fluid flow rate. Thus, retentate flow to the system outlet 19 may be controlled. In some implementations, the user or a controller 45 can selectively engage the second retentate valve 41 to direct retentate into the system outlet 19. Such routing of the retentate flow may be desirable where, in some implementations where cells are the target component to be extracted, data analysis identifies that the cells are a relatively high concentration of relatively high-proliferation cells that are ready for extraction from the system. In some implementations, the user or a controller 45 can selectively engage the second retentate valve 41 to obstruct retentate from flowing into the system outlet 19. Such obstruction of the retentate flow may be desirable where, again in some implementations where cells are the target component to be extracted, data analysis identifies a relatively high concentration of relatively low-proliferation cells (or a relatively low concentration of relatively high-proliferation cells), and further cell proliferation is desirable.

The bioprocessing system may further include a controller 45 operatively coupled to the first retentate flow line 22, the second retentate flow line 28, and the retentate system outlet flow line 26. The controller 45 may be configured to control a flow through each flow line 22, 26, 28. The controller 45 may control the flow through each line 22, 26, 28 to maintain a cell concentration of the contained bioprocessing environment 7. For example, in some applications, the cell concentration of the contained bioprocessing environment may be defined as the number of cells per unit volume. The cell concentration of the contained bioprocessing environment 7 may be maintained to remain substantially constant. Further, the cell concentration may be controlled to remain substantially constant when a cell product is removed from the bioprocessing system 9 by way of the retentate system outlet flow line 26. In alternative embodiments, when the controller 45 allows flow through the retentate system outlet flow line 26, the controller 45 does not allow flow through the second retentate flow line 28. Thus, cell product may be removed from the bioprocessing system 9 without affecting the cell concentration of the contained bioprocessing environment 7. This may advantageously allow continuous bioprocessing without interruption, which may be beneficial for various bioprocessing applications.

The bioprocessing system 9 may further include a bioprocessing flow line 34. The bioprocessing flow line is configured to fluidly couple the bioprocessing outlet 14 and the separator inlet 16. A feed pump FP can be configured for fluid communication with the bioprocessing flow line 34 to initiate, maintain, and/or regulate fluid flow through the line. A controller 45 can be in operative communication with the feed pump FP. In one or more embodiments, the bioprocessing flow line 34 may intersect with the first retentate flow line 22 prior to reaching the separator inlet 16 (FIG. 1). In alternative embodiments, the bioprocessing flow line 34 and the first retentate flow line 22 are both individually coupled to the separator inlet 16, which may include multiple separator inlets.

In some embodiments, the system 9 is adapted for data analysis of cell or other target biologic in the bioprocessing environment. One or more sensors 27 may be in communication with the fluid in the contained bioprocessing environment 7, or elsewhere in the bioprocessing system 9. Various individual sensors and combinations of sensors may be used, such as, for example, pH, conductivity, dissolved oxygen sensors, etc. The sensor may be configured to sense the fluid anywhere within the bioprocessing system 9. The controller 45 may be in communication with the one or more sensors and may be operatively coupled to the feed pump FP to direct fluid from the contained bioprocessing environment 7 to the separator assembly 6 upon meeting a parameter condition. For example, an ammonia sensor or a conductivity sensor may be in sensing communication with the fluid in the contained bioprocessing environment 7 and, upon sensing of a threshold level of ammonia in the fluid, the controller 45 can operate the feed pump FP to direct fluid to the separator assembly 6. Other types of sensing are certainly contemplated including sensing cell density. As another example, sensing data can be presented to a user, and the user can operate the feed pump FP to direct fluid flow to the separator assembly when appropriate.

In the current example, the contained bioprocessing environment 7 is in fluid communication with a culture media source 5. The culture media source 5 can be a source of fresh culture media, where “fresh” culture media refers to culture media substantially lacking cell byproduct. In some implementations the system is configured to selectively facilitate flow of culture media from the culture media source 5 to the contained bioprocessing environment 7. A valve and/or a pump (similar to those described elsewhere herein) can be used to selectively release culture media from the culture media source 5 to the contained bioprocessing environment 7. In some embodiments, the system 9 is configured to release culture media from the culture media source 5 to the contained bioprocessing environment 7 automatically. The system 9 can also be configured to stop culture media flow to the contained bioprocessing environment 7 automatically. For example, a sensor 27 disposed in sensing communication with the contained bioprocessing environment 7 may be in communication with the controller 45 that selectively directs and obstructs culture media flow from the culture media source 5 to the contained bioprocessing environment. In addition or alternatively to the sensor functions described above, the sensor 27 may also be configured to sense one or more of a fluid level, product concentration, or bi-product concentration, and the controller may selectively direct and obstruct culture media flow based on the sensor readings.

In one or more embodiments, the separator assembly 6 includes a diafiltration system. Diafiltration generally refers to a dilution process that involves the removal or separation of components of a solution based on their size by using permeable filtration media. In some implementations, diafiltration is used to exchange one or more components of the culture media, such a solution within the culture media. Diafiltration is generally a membrane filtration process. Diafiltration can be used to separate or remove small molecules, such as salts or solvents, from macromolecules, such as proteins or polymers, in a solution. Often a fresh solvent such as water or buffer is added to the retentate while simultaneously removing permeate, allowing for the selective removal of low-molecular-weight components based on size exclusion through a semipermeable membrane. Cells or other target biologic and media within the separator assembly 6 may be diluted in a washing fluid. The washing fluid may help remove undesirable components from the desirable components within the separator assembly 6. In alternative embodiments, the bioprocessing system 9 includes a washing line 36. The washing line 36 may be configured to fluidly couple a solution reservoir 2 and the separator inlet 16. The washing fluid may include one or more of: purified water, buffer material, and cell proliferation/growth media. In various embodiments, a washing fluid pump DP is configured to be in fluid communication with the washing line 36 to drive fluid flow through the washing line 36. In various embodiments a controller 45 is in communication with the washing fluid pump DP to control fluid flow through the washing line 36.

The bioprocessing system may further include a permeate line 24. In various implementations, a controller 45 is configured to regulate the flow rate and/or pressure of the permeate line. Such a configuration may advantageously allow regulation of the system to achieve a production cycle with relatively improved efficiency.

In one or more embodiments, the bioprocessing system 9 includes a sanitation system that is configured to clean and/or sterilize the system 9. The sanitation system may be configured to allow the bioprocessing system 9 to be cleaned-in-place. Clean-in-place (CIP) is a cleaning method that removes contaminants from the interior surfaces of equipment without taking the equipment apart. CIP may be automated in some implementations. CIP may use a mixture of water and chemicals, such as acids, alkalis, enzymes, ionic detergents, etc., that circulate to clean surfaces by removing substances on those surfaces and removing those substances from the system 9. In some embodiments the sanitation system is a sanitize-in-place system (SIP). A SIP system is a sanitizing method that destroys live organisms within the system. SIP systems can run steam through the system 9 to sanitize system components. In some embodiments, to allow the bioprocessing system to be CIP or SIP, the separation assembly 6 includes re-usable separation components that do not need to be replaced upon system cleaning/sanitization. Such a configuration may distinguish from existing bioprocessing systems that employ single use filters that are generally not cleaned and re-used but, rather, are replaced. Replacement of such filters introduces contamination risk that is avoided through the use of a separator system 6 having separator components that are reusable and cleanable within the bioprocessing system 9 absent removal from the system.

In various embodiments the fluid flow lines, separator assembly components, pumps, and the contained bioprocessing environment are configured to facilitate gravity-driven drainage of the system. Such a configuration further enables a CIP or SIP system.

The sanitation system can include various components and combinations of components. In some embodiments the sanitation system includes a heat exchanger. The heat exchanger can be configured to heat a cleaning solution. In some embodiments the sanitation system a regulation valve. In some embodiments the sanitation system includes a steam trap. In some embodiments the sanitation system includes a cleaning fluid reservoir, such as a tank containing a cleaning solution or a steam generator in communication with a liquid (e.g., water) source. In some embodiments the sanitation system includes various sensors in sensing communication with the fluid flow lines of the system that are configured to sense parameters associated with the fluid such as pressure, conductivity, temperature, and the like.

In various embodiments a controller (which may be the same controller 45 as discussed above or be a separate controller) is configured to execute a sanitation operation such as a CIP or SIP operation. The controller 45 can be configured to operate a pump to initiate sanitization fluid flow through the system, and selectively close and open valves within the system to selectively direct sanitization fluid through the valves, pumps, filters, and the like. In some embodiments the controller 45 is configured to operate a pump to reverse fluid flow through the system. In some embodiments the controller 45 is configured to keep the sanitizing system engaged until the controller 45 receives sensor data indicating that the sanitizing solution(s) are no longer in the system or sufficiently weak to prevent interference with bioprocessing system operation.

In various embodiments, the controller 45 is configured to regulate the sanitation system. The controller 45 can be in communication with one or more sensors that are configured to sense parameters relevant to the cleaning process, such as media temperature, pressure, pH, dissolved oxygen, carbon dioxide, and conductivity, as examples. Such parameters can provide an indication of the steps in the cleaning/sanitizing process and/or can correlate with the presence of cleaning chemicals remaining within the flow lines of the system. In some embodiments the controller 45 is configured to maintain the cleaning/sanitizing operation until the sensor parameters correlate with a lack of cleaning/sanitizing chemicals within the flow lines. The controller 45 can be in operative communication with each of the valves in the bioprocessing system to regulate and target the fluid flow through each of the flow lines. In some embodiments the controller 45 can be in communication with a pump that is configured to alternate the flow rate and/or reverse the fluid flow through the system.

The bioprocessing system 9 may further include a connecting flow line 50. The connecting flow line 50 may be fluidly coupled to the first retentate flow line 22 and the second retentate flow line 28. The connecting flow line 50 may be fluidly coupled to the first retentate flow line 22 and the retentate system outlet flow line 26. The connecting flow line 50 may advantageously allow for control of flow through the system with minimal static volume of fluid in a line that is not flowing.

In one or more embodiments, various valves may be included to assist in controlling flow through the system (e.g., the first retentate valve 40, the second retentate valve 41, and further valves as discussed herein). In some embodiments, the controller 45 may be in communication with one or more of the valves to regulate fluid flow through the system. For example, the bioprocessing system 9 may include a cell product valve 42 operably connected to the retentate system outlet flow line 26. The cell product valve 42 may be located on a bypass loop to regulate flow. Additionally, if the pressure in the bioprocessing system 9 is sufficient to extract the cell product without applying a pump, regulating flow using the cell product valve 42 without a pump may be beneficial for a shear-sensitive cell product. The bioprocessing system 9 may include a permeate valve 44 operably connected to the permeate line 24. The permeate valve 44 can be used to regulate fluid flow through the permeate line 24. In some embodiments the controller 45 is in operative communication with the permeate valve 44.

A loop breaker valve 21 can be disposed along the first retentate flow line 22 to selectively close the fluid flow loop between the separator inlet 16 and the separator outlet 18. Such a loop breaker valve 21 can be closed to block flow when the retentate flow is being directed to one or both of the contained bioprocessing environment 7 and the system outlet 19. In some embodiments one or more sensors can be installed in-line in sensing communication with the separator outlet 18 and, upon sensing of a threshold level of a contaminant, the loop breaker valve 21 can be opened and the first retentate valve 40 and the cell product valve 42 can be closed (either automatically by a controller 45 in communication with the sensor or by a user who observes the sensor reading) to cycle the retentate through the separation assembly 6. In such an example, upon sensing a contaminant is below a threshold, the first retentate valve 40 and/or the cell product valve 42 can be selectively opened to direct retentate to the contained bioprocessing environment 7 or the system outlet. In some such implementations it may be desirable to close the loop breaker valve 21.

Further, for example, in one or more applications, the loop breaker valve 21 may be continuously open to allow retentate to recirculate through the separator assembly 6. In one or more applications, the loop breaker valve 21 may be intermittently open and closed to allow and block, respectively, retentate recirculation through the separator assembly. In one or more applications, the loop breaker valve 21 may be continuously closed to block retentate recirculation through the separator assembly. In one or more embodiments, the loop breaker valve 21 may be omitted. In such embodiments, there may be a flow line (similar to a continuously open valve) or there may be no flow line (similar to a continuously closed valve).

When the retentate recirculation through the separator assembly is blocked in any embodiment, the retentate may flow to the bioprocessing environment 7, the system outlet 19, or both the bioprocessing environment 7 and the system outlet 19.

The bleed pump BP and the loop pump LP may be used to control the flow rate of the retentate in any embodiment, in order to achieve constant or substantially constant or about constant cell concentration of the bioprocessing environment 7. For example, in embodiments where the loop breaker valve 21 is always open or always closed, the bleed pump BP and the loop pump LP may each maintain a constant or substantially constant or about constant flow rate through the retentate system outlet flow line 26 and the second retentate flow line 28, respectively. Further, for example, in embodiments where the loop breaker valve 21 is intermittently open and closed, the bleed pump BP and the loop pump LP may each set a variable flow rate through the retentate system outlet flow line 26 and the second retentate flow line 28, respectively. The first retentate flow line 22, the second retentate flow line 28, the retentate system outlet flow line 26, the bleed pump BP, the loop pump LP, the loop breaker valve 21, the first retentate valve 40, the second retentate valve 41, and the cell product valve 42 may be individually and collectively controlled to achieve constant or substantially constant or about constant cell concentration of the bioprocessing environment 7.

In some embodiments, the first retentate flow line 22, the second retentate flow line 28, the retentate system outlet flow line 26, the bleed pump BP, the loop pump LP, the loop breaker valve 21, the first retentate valve 40, the second retentate valve 41, and the cell product valve 42 may be individually and collectively controlled to achieve flow from the bioprocessing environment 7 through a portion of the first retentate flow line 22 such that the flow bypasses the separator assembly 6, and towards the second retentate flow line 28 and/or towards the retentate system outlet flow line 26 and/or towards the connecting flow line 50. The bleed pump BP and the loop pump LP may maintain a constant flow rate through the portion of the first retentate flow line 22, or may provide a variable flow rate therethrough.

Various pressure sensors may be included in the system. In some embodiments the controller 45 is in communication with each of the pressure sensors to regulate flow through the system in response to the sensed pressure. Pressure sensors P and flow sensors F can be in sensing communication with the various flow lines in the system 9. In some embodiments, the pressure sensors P and flow sensors F are configured to transmit such data to a user interface and/or a controller 45. Such data can be used by the controller 45 and/or a user to adjust fluid flow through the system 9.

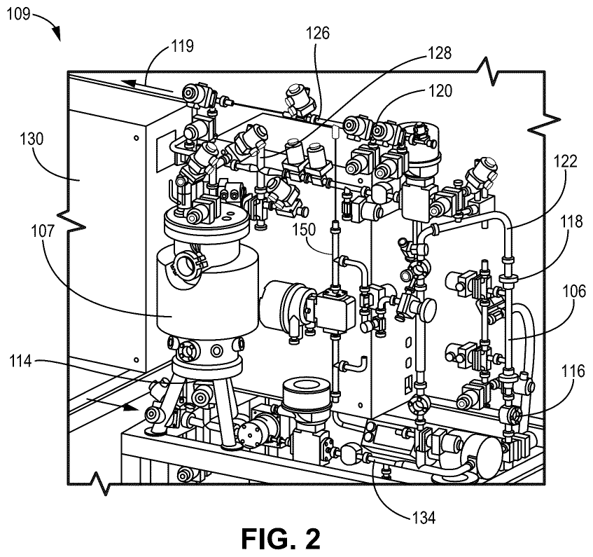

FIG. 2 illustrates a diagram of a bioprocessing system 109. The system 109 may include elements similar to those depicted and described with respect to the system 9 of FIG. 1.

The bioprocessing system 109 has a separator assembly 106 having a separator inlet 116, a retentate outlet 118 and a permeate outlet 120. The discussions of the components depicted above applies to components depicted here except where contrary to the current description or figure. In the current example, the contained bioprocessing environment (such as a bioreactor) is omitted, although the system is configured to be in fluid communication with a bioreactor, such as along retentate flow line 126 and bioprocessing flow line 134.

A first retentate flow line 122 is configured to be fluidly coupled to the retentate outlet 118 and the separator inlet 116. A second retentate flow line 126 is configured to be fluidly coupled to the retentate outlet 118 and the contained bioprocessing environment. A retentate system outlet flow line 150 is configured to be fluidly coupled to the retentate outlet and a system outlet.

In this example the system has a sanitation system including a CIP tank 107 and a CIP tank switch valve in operative communication with the CIP tank 107. Also in this example, a controller 130 is in sensing and operative communication with various system components. The system can include various valves, pumps, and sensors that have been described in detail above.

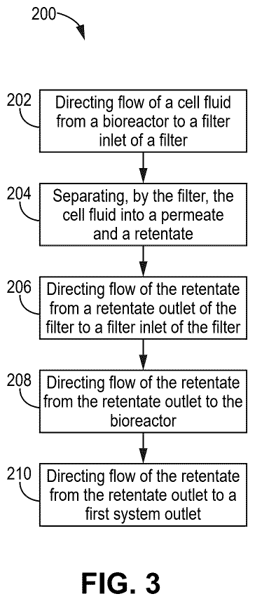

In one or more embodiments, a method 200 (FIG. 3) for operating a bioprocessing system such as the systems 9, 109 described herein may include, in no particular order: directing flow of a fluid (e.g., culture media) from a contained bioprocessing environment 7, 107 to a separator inlet 16, 116 of a separator assembly 6, 106 (step 202), separating, by the separator assembly 6, 106, the fluid into a permeate and a retentate (step 204), directing flow of the retentate from a retentate outlet 18, 118 of the separator assembly 6, 106 to a separator inlet 16, 116 of the separator assembly 6, 106 (step 206), directing flow of the retentate from the retentate outlet 18, 118 to the contained bioprocessing environment 7, 107 (step 208), and directing flow of the retentate from the retentate outlet 18, 118 to a first system outlet 19, 119 (step 210).

Directing flow of the retentate from the retentate outlet 18, 118 to the separator inlet 16, 116 (step 206) may further include directing flow through a first retentate flow line 22, 122. Directing flow of the retentate from the retentate outlet 18, 118 to the separator inlet 16, 116 bypasses the contained bioprocessing environment 7, 107 and the first system outlet 19, 119. In such embodiments, retentate can flow directly to the separator inlet 16, 116 from the retentate outlet 18, 118. Directing flow of the retentate from the retentate outlet 18, 118 to the contained bioprocessing environment 7, 107 may further include directing flow through a second retentate flow line 28, 128. Directing flow of the retentate from the retentate outlet 18, 118 to the contained bioprocessing environment 7, 107 bypasses the separator inlet 16, 116 and the first system outlet 19, 119. In such embodiments, retentate can flow directly to the contained bioprocessing environment 7, 107 from the retentate outlet 18, 118. Directing flow of the retentate from the retentate outlet 18, 118 to a first system outlet 19, 119 may further include directing flow through a retentate system outlet flow line 26, 126. Directing flow of the retentate from the retentate outlet 18, 118 to the first system outlet 19, 119 bypasses the separator inlet 16, 116 and the contained bioprocessing environment 7, 107. In such embodiments, retentate can flow directly to the first system outlet 19, 119 from the retentate outlet 18, 118. In some embodiments flow of the retentate can be directed to two or more of the first system outlet 19, 119, the contained bioprocessing environment 7, 107, and the separator inlet 16, 116 simultaneously. In some embodiments, flow of the retentate can be directed to each of the first system outlet 19, 119, the contained bioprocessing environment 7, 107, and the separator inlet 16, 116 simultaneously.

The method 200 may further include controlling a retentate valve operatively coupled to the second retentate flow line 28, 128 using a controller operatively coupled to the retentate valve. The method 200 may further include directing flow from the contained bioprocessing environment 7, 107 to the separator inlet 16, 116. The method 200 may further include diafiltrating the fluid using the separator assembly 6, 106. The method 200 may further include directing flow of a washing fluid from a washing line 36, 136 to the separator inlet 16, 116. The method 200 may further include directing flow of the permeate from a permeate outlet 20, 120 of the separator assembly 6, 106 to the washing line 36, 136 (not shown). The method 200 may further include maintaining a cell concentration such that the cell concentration remains substantially constant of the contained bioprocessing environment 7, 107 when a cell product is removed from the system outlet 19, 119. The method 200 may further include initiating a clean-in-place system cleaning procedure.

In one or more embodiments, when a cell product is removed from the bioprocessing system 9, 109, volumetric production rate in the contained bioprocessing environment 7, 107 is not affected by the removal of the cell product. Volumetric production rate refers to cell proliferation in some implementations, where the cell proliferation rate is quantified by unit volume. Volumetric production rate can also refer to cell productivity, which quantifies the cell productivity rate by unit volume. By “not affected” it is meant that that volumetric productivity does not decrease by more than 50%, 40%, 30%, 20%, 10%, 5%, 3%, or 1%. Such a configuration enables continuous production of the cell products.

The method 200 may further include operating a controller operably coupled to the bioprocessing system 9, 109 to control a cell proliferation phase of cells in the contained bioprocessing environment 7, 107. The cell proliferation phase of the cells may be controlled or configured to remain at or above 4 log proliferation after reaching 4 log proliferation. In alternative embodiments, the cell proliferation phase of the cells is configured to remain at or above 7 log proliferation after reaching 7 log proliferation. In various implementations the system is configured to maintain proliferation between 4 log proliferation and 7 log proliferation.

### Exemplary Aspects

Aspect 1. A bioprocessing system comprising:
a separator assembly comprising a separator inlet, a retentate outlet, and a permeate outlet, wherein the separator assembly is configured to be fluidly coupled to a contained bioprocessing environment via a bioprocessing environment flow line;
a first retentate flow line configured to be fluidly coupled to the retentate outlet and the separator inlet;
a second retentate flow line configured to be fluidly coupled to the retentate outlet and the contained bioprocessing environment; and
a retentate system outlet flow line configured to be fluidly coupled to the retentate outlet and a system outlet.
Aspect 2. The bioprocessing system of any one of Aspects 1 and 3-23, further comprising a retentate valve operatively coupled to the second retentate flow line.
Aspect 3. The bioprocessing system of any one of Aspects 1-2 and 4-23, wherein the separator assembly comprises a diafiltration system.
Aspect 4. The bioprocessing system of any one of Aspects 1-3 and 5-23, further comprising a washing line fluidly coupled to the separator inlet.
Aspect 5. The bioprocessing system of any one of Aspects 1-4 and 6-23, wherein the bioprocessing system is configured to be cleaned-in-place.
Aspect 6. The bioprocessing system of any one of Aspects 1-5 and 7-23, wherein the separator assembly comprises components that are reusable and washable.
Aspect 7. The bioprocessing system of any one of Aspects 1-6 and 8-23, wherein the separator assembly excludes single use components.
Aspect 8. The bioprocessing system of any one of Aspects 1-7 and 9-23, further comprising a cleaning solution tank in selective communication with the separation assembly.
Aspect 9. The bioprocessing system of any one of Aspects 1-8 and 10-23, further comprising a steam generator configured for selective fluid communication with the cleaning solution tank.
Aspect 10. The bioprocessing system any one of Aspects 1-9 and 11-23, wherein the separator assembly comprises a tangential flow filter.
Aspect 11. The bioprocessing system of any one of Aspects 1-10 and 12-23, wherein the separator assembly comprises a hydrodynamic separator.
Aspect 12. The bioprocessing system of any one of Aspects 1-11 and 13-23, wherein the separator assembly comprises a membrane.
Aspect 13. The bioprocessing system of any one of Aspects 1-12 and 14-23, further comprising a controller operatively coupled to the first retentate flow line, the second retentate flow line, and the retentate system outlet flow line, and configured to control a flow through each flow line.
Aspect 14. The bioprocessing system of any one of Aspects 1-13 and 15-23, wherein the controller is configured to control the flow through each line to maintain a cell concentration of the contained bioprocessing environment, and wherein the cell concentration of the contained bioprocessing environment remains substantially constant when a cell product is removed from the bioprocessing system by way of the retentate system outlet flow line.
Aspect 15. The bioprocessing system of any one of Aspects 1-14 and 16-23, wherein, when the controller allows flow through the retentate system outlet flow line, the controller does not allow flow through the second retentate flow line.
Aspect 16. The bioprocessing system of any one of Aspects 1-15 and 17-23, further comprising a connecting flow line, wherein the connecting flow line is fluidly coupled to the first retentate flow line and the second retentate flow line, and wherein the connecting flow line is fluidly coupled to the first retentate flow line and the retentate system outlet flow line.
Aspect 17. The bioprocessing system of any one of Aspects 1-16 and 18-23, further comprising a connecting line valve operably coupled to the connecting flow line and controllable to open or close the connecting flow line.
Aspect 18. The bioprocessing system of any one of Aspects 1-17 and 19-23, wherein the first retentate flow line, the second retentate flow line, and the retentate system outlet flow line are each fluidly coupled to a four-way connector, and wherein the four-way connector is fluidly coupled to the retentate outlet.
Aspect 19. The bioprocessing system of any one of Aspects 1-18 and 20-23, further comprising the contained bioprocessing environment.
Aspect 20. The bioprocessing system of any one of Aspects 1-19 and 21-23, wherein the contained bioprocessing environment comprises a bioreactor.
Aspect 21. The bioprocessing system of any one of Aspects 1-20 and 22-23, wherein the contained bioprocessing environment is configured for fluid communication with the second retentate flow line, wherein fluid flow from the contained bioprocessing environment is configured to bypass the separator assembly.
Aspect 22. The bioprocessing system of any one of Aspects 1-21 and 23, wherein the system is configured for continuous bioprocessing.
Aspect 23. The bioprocessing system of any one of Aspects 1-22, wherein the system is configured to continuous collection of cell product.
Aspect 24. A method for operating a bioprocessing system, the method comprising: directing flow of a fluid from a contained bioprocessing environment to a separator inlet of a separator assembly;
separating, by the separator assembly, the fluid into a permeate and a retentate;
selectively directing flow of the retentate from a retentate outlet of the separator assembly to two or more in the group consisting of:
   a separator inlet of the separator assembly;
   the contained bioprocessing environment; and
   a first system outlet.
Aspect 25. The method of any one of Aspects 24 and 26-41, wherein directing flow of the retentate from the retentate outlet to the separator inlet comprises directing flow through a first retentate flow line.
Aspect 26. The method of any one of Aspects 24-25 and 27-41, wherein directing flow of the retentate from the retentate outlet to the contained bioprocessing environment comprises directing flow through a second retentate flow line.
Aspect 27. The method of any one of Aspects 24-26 and 28-41, further comprising controlling a retentate valve operatively coupled to the second retentate flow line using a controller operatively coupled to the retentate valve.
Aspect 28. The method of any one of Aspects 24-27 and 29-41, wherein directing flow of the retentate from the retentate outlet to a first system outlet comprises directing flow through a retentate system outlet flow line.
Aspect 29. The method of any one of Aspects 24-28 and 30-41, further comprising directing flow from the contained bioprocessing environment to the separator inlet.
Aspect 30. The method of any one of Aspects 24-29 and 31-41, further comprising directing flow from the contained bioprocessing environment through the second retentate flow line and bypassing the separator assembly.
Aspect 31. The method of any one of Aspects 24-30 and 32-41, further comprising diafiltrating the fluid using the separator assembly.
Aspect 32. The method of any one of Aspects 24-31 and 33-41, further comprising directing flow of a washing fluid from a washing line to the separator inlet.
Aspect 33. The method of any one of Aspects 24-32 and 34-41, further comprising directing flow of the permeate from a permeate outlet of the separator assembly to the washing line.
Aspect 34. The method of any one of Aspects 24-33 and 35-41, further comprising maintaining a cell concentration of the contained bioprocessing environment when a cell product is removed from the first system outlet.
Aspect 35. The method of any one of Aspects 24-34 and 36-41, wherein, when a cell product is removed from the bioprocessing system, volumetric production rate is not affected by the removal of the cell product.
Aspect 36. The method of any one of Aspects 24-35 and 37-41, further comprising initiating a clean-in-place system cleaning procedure.
Aspect 37. The method of any one of Aspects 24-36 and 38-41, wherein the separator assembly comprises a tangential flow filter.
Aspect 38. The method of any one of Aspects 24-37 and 39-41, wherein the separator assembly comprises a hydrodynamic separator.
Aspect 39. The method of any one of Aspects 24-38 and 40-41, wherein the separator assembly comprises a membrane separator.
Aspect 40. The method of any one of Aspects 24-39 and 41, wherein the method is a substantially continuous bioprocessing method.
Aspect 41. The method of any one of Aspects 24-40, further comprising substantially continuous collection of cell product.
Aspect 42. A bioprocessing system comprising:
a contained bioprocessing environment;
a separator assembly comprising a separator inlet, a retentate outlet, and a permeate outlet, wherein the separator inlet is configured for fluid communication with the contained bioprocessing environment;
a first retentate flow line configured to be fluidly coupled to the retentate outlet and the separator inlet;
a second retentate flow line configured to be fluidly coupled to the first retentate flow line and the contained bioprocessing environment;
a retentate system outlet flow line configured to be fluidly coupled to the first retentate flow line and a system outlet;
a connecting flow line, wherein the connecting flow line is configured to be fluidly coupled to the first retentate flow line, the second retentate flow line, and the retentate system outlet flow line;
a loop breaker valve operatively coupled to the first retentate flow line;
a first retentate valve operatively coupled to the second retentate flow line; and
a second retentate valve operatively coupled to the retentate system outlet flow line, whereby bypassing the separator assembly is enabled.
Aspect 43. The bioprocessing system of any one of Aspects 42 and 44-46, wherein the loop breaker valve is positioned downstream of the connection flow line.
Aspect 44. The bioprocessing system of any one of Aspects 42-43 and 45-46, wherein each of the loop breaker valve, first retentate valve, and the second retentate valve have a fully open position, a fully closed position, and an intermediate position between the fully open and fully closed position.
Aspect 45. The bioprocessing system of any one of Aspects 42-44 and 46, wherein the system is configured for continuous bioprocessing.
Aspect 46. The bioprocessing system of any one of Aspects 42-45, wherein the system is configured for continuous collection of cell product.

It should be noted that, as used in this specification and the appended claims, the phrase “configured” describes a system, apparatus, or other structure that is constructed to perform a particular task or adopt a particular configuration. The word “configured” can be used interchangeably with similar words such as “arranged”, “constructed”, “manufactured”, and the like.

The term “about” as used herein can allow for a degree of variability in a value or range, for example, within 10%, within 5%, or within 1% of a stated value or of a stated limit of a range and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

All publications and patent applications in this specification are indicative of the level of ordinary skill in the art to which this technology pertains. All publications and patent applications are herein incorporated by reference to the same extent as if each individual publication or patent application was specifically and individually indicated by reference. In the event that any inconsistency exists between the disclosure of the present application and the disclosure(s) of any document incorporated herein by reference, the disclosure of the present application shall govern.

This application is intended to cover adaptations or variations of the present subject matter. It is to be understood that the above description is intended to be illustrative, and not restrictive, and the claims are not limited to the illustrative embodiments as set forth herein.

What is claimed is:

1. A bioprocessing system comprising:
a separator assembly comprising a separator inlet, a retentate outlet, and a permeate outlet, wherein the separator assembly is configured to be fluidly coupled to a contained bioprocessing environment via a bioprocessing environment flow line;
a first retentate flow line configured to be fluidly coupled to the retentate outlet and the separator inlet;
a second retentate flow line configured to be fluidly coupled to the retentate outlet and the contained bioprocessing environment; and
a retentate system outlet flow line configured to be fluidly coupled to the retentate outlet and a system outlet.

2. The bioprocessing system of claim 1, further comprising a retentate valve operatively coupled to the second retentate flow line.

3. The bioprocessing system of claim 1, wherein the separator assembly comprises a diafiltration system.

4. The bioprocessing system of any one of claim 1, further comprising a washing line fluidly coupled to the separator inlet.

5. The bioprocessing system of claim 1, wherein the bioprocessing system is configured to be cleaned-in-place.

6. The bioprocessing system of claim 1, further comprising a cleaning solution tank in selective communication with the separator assembly.

7. The bioprocessing system of claim 6, further comprising a steam generator configured for selective fluid communication with the cleaning solution tank.

8. The bioprocessing system of claim 1, further comprising a controller operatively coupled to the first retentate flow line, the second retentate flow line, and the retentate system outlet flow line, and configured to control a flow through each flow line.

9. The bioprocessing system of claim 8, wherein the controller is configured to control the flow through each line to maintain a cell concentration of the contained bioprocessing environment, and wherein the cell concentration of the contained bioprocessing environment remains substantially constant when a cell product is removed from the bioprocessing system by way of the retentate system outlet flow line.

10. The bioprocessing system of claim 1, further comprising a connecting flow line, wherein the connecting flow line is fluidly coupled to the first retentate flow line and the second retentate flow line, and wherein the connecting flow line is fluidly coupled to the first retentate flow line and the retentate system outlet flow line.

11. The bioprocessing system of claim 10, further comprising a connecting line valve operably coupled to the connecting flow line and controllable to open or close the connecting flow line.

12. The bioprocessing system of claim 1, wherein the first retentate flow line, the second retentate flow line, and the retentate system outlet flow line are each fluidly coupled to a four-way connector, and wherein the four-way connector is fluidly coupled to the retentate outlet.

13. The bioprocessing system of claim 1, further comprising the contained bioprocessing environment, which optionally comprises a bioreactor.

14. The bioprocessing system of claim 1, wherein the contained bioprocessing environment is configured for fluid communication with the second retentate flow line, wherein fluid flow from the contained bioprocessing environment is configured to bypass the separator assembly.

15. A method for operating a bioprocessing system, the method comprising:
directing flow of a fluid from a contained bioprocessing environment to a separator inlet of a separator assembly;
separating, by the separator assembly, the fluid into a permeate and a retentate;
selectively directing flow of the retentate from a retentate outlet of the separator assembly to two or more in the group consisting of:
a separator inlet of the separator assembly;
the contained bioprocessing environment; and
a first system outlet.

16. The method of claim 15, further comprising maintaining a substantially constant cell concentration of the contained bioprocessing environment when a cell product is removed from the first system outlet.

17. The method of claim 15, wherein, when a cell product is removed from the bioprocessing system, volumetric production rate is not affected by the removal of the cell product.

18. The method of claim 15, wherein the method is a substantially continuous bioprocessing method.

19. The method of claim 15, further comprising substantially continuous collection of cell product.

20. A bioprocessing system comprising:
a contained bioprocessing environment;
a separator assembly comprising a separator inlet, a retentate outlet, and a permeate outlet, wherein the separator inlet is configured for fluid communication with the contained bioprocessing environment;
a first retentate flow line configured to be fluidly coupled to the retentate outlet and the separator inlet;
a second retentate flow line configured to be fluidly coupled to the first retentate flow line and the contained bioprocessing environment;
a retentate system outlet flow line configured to be fluidly coupled to the first retentate flow line and a system outlet;
a connecting flow line, wherein the connecting flow line is configured to be fluidly coupled to the first retentate flow line, the second retentate flow line, and the retentate system outlet flow line;
a loop breaker valve operatively coupled to the first retentate flow line;
a first retentate valve operatively coupled to the second retentate flow line; and
a second retentate valve operatively coupled to the retentate system outlet flow line, whereby bypassing the separator assembly is enabled.
